(19) United States
(12) Patent Application Publication
(10) Pub. No.: US 2023/0183154 A1
(43) Pub. Date: Jun. 15, 2023

(54) PROCESS FOR THE PREPARATION OF 1-BROMO-2,4,5-TRIFLUOROBENZENE

(71) Applicant: AARTI INDUSTRIES LIMITED, Mumbai (IN)

(72) Inventors: Mirik GOGRI, Mumbai (IN); Prashant POTNIS, Mumbai (IN); Surender DHINGRA, Mumbai (IN)

(21) Appl. No.: 17/906,059

(22) PCT Filed: Mar. 10, 2021
(86) PCT No.: PCT/IN2021/050235

§ 371 Date: Sep. 9, 2022

(30) Foreign Application Priority Data
Mar. 12, 2020 (IN) 202021010637

(51) Int. Cl. C07C 17/14

(52) U.S. Cl. CPC C07C 17/14

(57) ABSTRACT

A process for the preparation of 1-bromo-2,4,5-trifluorobenzene from 2,4,5-trifluoroaniline or sulfate salt thereof.

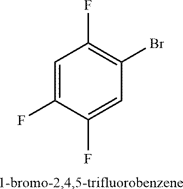

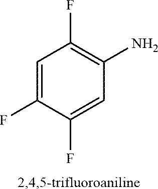

A process for the preparation of 1 ,2,4-trifluorobenzene from 2,4,5-trifluoroaniline and then converting into 1-bromo-2,4,5-trifluorobenzene.

### FIELD OF THE INVENTION

The present invention relates to a novel process for the preparation of 1-bromo-2,4,5 -trifluorobenzene.

### BACKGROUND

1-bromo-2,4,5-trifluorobenzene is a key intermediate for the synthesis of several pharmaceutical, agrochemical, and other important fine chemical products.

For example, quinolone, an antimicrobial agrochemical product, is prepared using 1-bromo-2,4,5-trifluorobenzene.

1-bromo-2,4,5-trifluorobenzene is useful for variety of antimicrobial drugs such as ciprofloxacin, moxifloxacin, gatifloxacin and pefloxacin. 1-bromo-2,4,5-trifluorobenzene is also a crucial intermediate in the synthesis of sitagliptin, a drug for treatment of type 2 diabetes mellitus.

The preparation of 1-bromo-2,4,5-trifluorobenzene by bromination of 1,2,4-trifluorobenzene is reported in Chinese patent CN101168495 and Indian patent application 92/DEL/2015.

A Chinese patent CN101168495 discloses a process wherein 1,2,4-trifluorobenzene is reacted with elemental bromine using iron powder and a radical initiator such as azobisisobutyronitrile in the presence of an organic solvent such as chloroform or carbon tetrachloride to obtain 1-bromo-2,4,5-trifluorobenzene. The process disclosed in CN101168495 is tedious involving numerous steps and gives a product with a low yield. Further, the solvents used therein are chlorinated solvents which are not suitable for large scale industrial production.

An Indian patent application 92/DEL/2015 also discloses a similar process wherein 1,2,4-trifluorobenzene is reacted with elemental bromine using catalytic iron powder or ferric chloride to obtain 1-bromo-2,4,5-trifluorobenzene in good yield.

An article titled “The continuous kilogram-scale process for the synthesis of 2,4,5-trifluorobromobenzene via Gattermann reaction using micro-reactors” by Qiulin Deng et. al. discloses the synthesis of 1-bromo-2,4,5-trifluorobenzene from 2,4,5-trifluoroaniline. 2,4,5-trifluoroaniline is diazotized using sodium nitrite and hydrogen bromide to obtain 2,4,5-trifluorophenyl diazonium salt which is then reacted with aqueous hydrogen bromide in the presence of copper powder to obtain 1-bromo-2,4,5-trifluorobenzene.

The shortcomings of this process are that it is a flow process involving a typically arranged model of micro and batch reactors. The process disclosed by Deng et. al. not only requires a capital investment for flow reactors but also demands a special operational expertise making its commercial implementation difficult.

Also, in the batch process, when 2,4,5-trifluoroaniline is diazotized using sodium nitrite and hydrogen bromide followed by bromination using aqueous hydrogen bromide and copper (I) bromide, the complete reaction mass gets polymerized.

In view of the above, a number of processes are available for the preparation of 1-bromo-2,4,5-trifluorobenzene, which have their own advantages and disadvantages. Still there is a continuing need to develop alternative processes for the manufacture of 1-bromo-2,4,5-trifluorobenzene. Specifically, there is a need in the art to develop a process for the preparation of 1-bromo-2,4,5-trifluorobenzene, which obviates prior problems, such as lower yield, tedious process, special operational expertise and polymerization of reaction mass.

The inventors of the present invention envisaged novel processes for the preparation of 1-bromo-2,4,5-trifluorobenzene which are simple and amenable for commercial production.

### OBJECTS OF THE INVENTION

Some of the objects of the present invention are described herein below:

It is an object of the present invention to ameliorate one or more problems of the prior art or to at least provide a useful alternative.

An object of the present invention is to provide processes for the preparation of 1-bromo-2,4,5-trifluorobenzene from 2,4,5-trifluoroaniline or sulfate salt thereof which employs reagents suitable for large scale batch production.

Another object of the present invention is to provide processes for the preparation of 1-bromo-2,4,5-trifluorobenzene from 2,4,5-trifluoroaniline or sulfate salt thereof in which reaction temperature is easily controlled.

Another object of the present invention is to provide processes for the preparation of 1-bromo-2,4,5-trifluorobenzene from 2,4,5-trifluoroaniline or sulfate salt thereof in which the formation of the polymerized product or polymerization of the reaction mass is obviated.

Yet another object of the present invention is to provide processes for the preparation of 1-bromo-2,4,5-trifluorobenzene from 2,4,5-trifluoroaniline or sulfate salt thereof which aims to obviate thermal decomposition of the diazotized intermediates.

Yet another object of the present invention is to provide processes for the preparation of 1-bromo-2,4,5-trifluorobenzene in which 1,2,4-trifluorobenzene is obtained from 2,4,5-trifluoroaniline.

Other objects and advantages of the present invention will be more apparent from the following description which is not intended to limit the scope of the present invention.

### SUMMARY OF THE INVENTION

The present invention relates to process for the preparation of 1-bromo-2,4,5-trifluorobenzene. The process comprises of converting 2,4,5-trifluoroaniline or a sulfate salt thereof into an intermediate. The intermediate is formed by reacting 2,4,5-trifluoroaniline or a sulfate salt thereof with nitrosulphuric acid. The intermediate can also be formed by reacting 2,4,5-trifluoroaniline with sodium nitrite in the presence of mineral acid and per acid. Subsequently, the intermediate is brominated to obtain 1-bromo-2,4,5-trifluorobenzene.

The present invention also relates to a process for preparation of an intermediate, 2,4,5-trifluorophenyl diazonium salt or 1,2,4-trifluorobenzene, to prepare 1-bromo-2,4,5-trifluorobenzene. The process involves converting 2,4,5-trifluoroaniline or sulfate salt thereof into an intermediate. The intermediate is formed either by reacting 2,4,5-trifluoroaniline or a sulfate salt thereof with nitrosulphuric acid to form 2,4,5-trifluorophenyl diazonium salt or by reacting 2,4,5-trifluoroaniline with sodium nitrite in the presence of mineral acid and per acid to form 1,2,4-trifluorobenzene.

### DETAILED DESCRIPTION OF THE INVENTION

The present invention discloses a process for preparation of 1-bromo-2,4,5-trifluorobenzene. The first step involves converting 2,4,5-trifluoroaniline or a sulfate salt thereof into an intermediate. The intermediate is formed by either of the following processes -
1. The intermediate is formed by reacting 2,4,5-trifluoroaniline or a sulfate salt thereof with nitrosulphuric acid.
2. The intermediate is formed by by reacting 2,4,5-trifluoroaniline with sodium nitrite in the presence of mineral acid and per acid.

The formation of intermediate is followed by bromination to obtain 1-bromo-2,4,5 -trifluorobenzene.

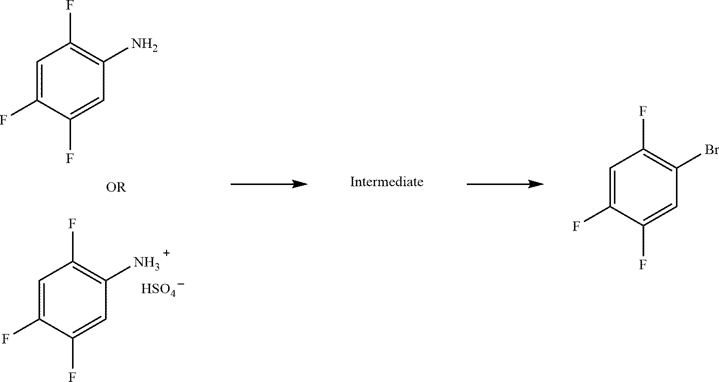

The present invention in one aspect provides processes for the preparation of 1-bromo-2,4,5-trifluorobenzene from 2,4,5-trifluoroaniline or sulfate salt thereof.

The process for the preparation of 1-bromo-2,4,5-trifluorobenzene in one embodiment of the present invention is described herein after.

In the first step, 2,4,5-trifluoroaniline or sulfate salt thereof is diazotized using nitrosulphuric acid to obtain 2,4,5-trifluorophenyl diazonium salt.

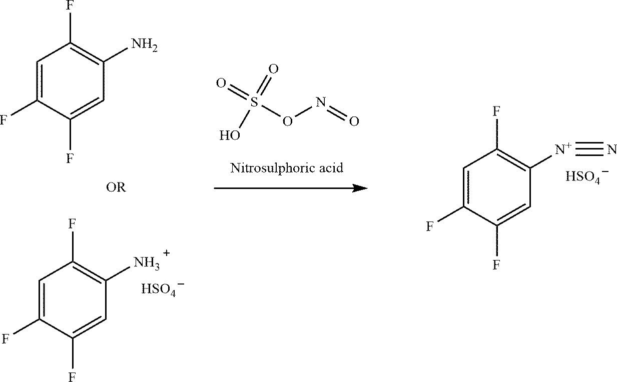

Subsequently, 2,4,5-trifluorophenyl diazonium salt is reacted with a suitable brominating agent in the presence of a catalytic amount of a suitable metal bromide to obtain 1-bromo-2,4,5-trifluorobenzene.

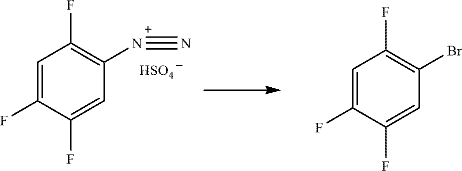

Typically, the metal bromide is added or generated in-situ to obtain crude 1-bromo-2,4,5-trifluorobenzene.

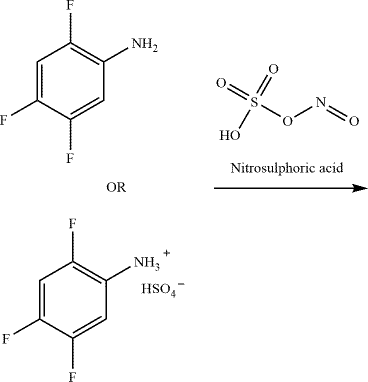

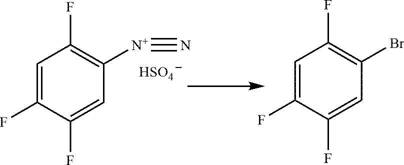

Though the purity of 2,4,5-trifluoroaniline or sulfate salt thereof will not affect the reaction conversion, yield, purity of the product and the like it is desirable to have more than 90% purity of 2,4,5-trifluoroaniline or sulfate salt thereof. Typically, the purity of 2,4,5-trifluoroaniline or sulfate salt thereof can range from 92% to 98% by HPLC.

The diazotization reaction is carried out optionally in the presence of a solvent.

The diazotization reaction typically is carried out at a temperature ranging from 0° C. to 50° C., preferably at a temperature ranging from 0° C. to 40° C., more preferably at a temperature ranging from 15° C. to 40° C.

The diazotization reaction is carried out for 5 seconds to 12 hours, preferably the reaction is carried out for 5 seconds to 5 hours.

Nitrosulphuric acid used for the diazotization reaction is 10% to 40% by weight nitrosulphuric acid in sulphuric acid, preferably 25% to 40% by weight nitrosulphuric acid in sulphuric acid, more preferably 30% to 40% by weight nitrosulphuric acid in sulphuric acid.

Molar ratio of 2,4,5-trifluoroaniline: nitrosulphuric acid for the diazotization reaction is selected from the range of 1:1 to 1:1.5.

Molar ratio of 2,4,5-trifluoroaniline sulfate: nitrosulphuric acid for the diazotization reaction is selected from the range of 1:1 to 1:1.5.

After completion of diazotization reaction, subsequently 2,4,5-trifluorophenyl diazonium salt is reacted with a suitable brominating agent in the presence of catalytic amount of a suitable metal bromide to obtain 1-bromo-2,4,5-trifluorobenzene.

The intermediate 2,4,5-trifluorophenyl diazonium salt may or may not be isolated. Particularly, 2,4,5-trifluorophenyl diazonium salt is not isolated.

Non-limiting examples of brominating agent suitable for brominating 2,4,5-trifluorophenyl diazonium salt include hydrogen bromide, elemental bromine, N-bromosuccinimide and dibromoisocyanuric acid, preferably the brominating agent is hydrogen bromide or elemental bromine.

Hydrogen bromide is used as an aqueous hydrogen bromide having concentrations of 10% to 48% by weight, preferably 18% to 48% by weight is used.

Alternatively, 33% by weight hydrogen bromide in acetic acid can also be used as a brominating agent.

Non-limiting examples of metal bromide suitable for the reaction of brominating 2,4,5-trifluorophenyl diazonium salt include copper (I) bromide and copper (II) bromide.

Metal bromides either alone or in combination with copper (I) bromide or copper (II) bromide that is suitable for brominating 2,4,5-trifluorophenyl diazonium salt include aluminum bromide, barium bromide, boron tribromide, cesium bromide, chromium bromide, cobalt bromide, dysprosium bromide, iron (II) bromide, iron (III) bromide, lithium bromide, magnesium bromide, phosphorus bromide, potassium bromide, sodium bromide, tin bromide, titanium bromide and zinc bromide. Preferably, the metal bromide is selected from copper (I) bromide and copper (II) bromide.

Alternatively, copper bromide used for brominating the 2,4,5-trifluorophenyl diazonium salt can also be prepared in-situ during reaction, wherein in-situ generated metal bromide is formed in-situ by reacting copper or cupric oxide with the brominating agent selected from hydrogen bromide or bromine.

Metal bromide is used in the amount of 1.0% to 10.0% by weight with respect to 2,4,5-trifluoroaniline or sulfate salt thereof, preferably 2.5% to 3.0% by weight metal bromide is used with respect to 2,4,5-trifluoroaniline or sulfate salt thereof.

The bromination reaction is carried out in the absence of a solvent.

The bromination reaction is carried out in the presence of a suitable solvent. Non-limiting examples of solvents suitable for the bromination reaction include nitriles, acetic acid, dimethyl sulfoxide, water and mixtures thereof. Preferably, the nitrile is acetonitrile.

The bromination reaction typically is carried out at a temperature ranging from 0° C. to 150° C., preferably at a temperature ranging from 40° C. to 110° C.

The bromination reaction is carried out for 0.5 hour to 12 hours, preferably for 1 hour to 7 hours.

After completion of bromination reaction, reaction mixture is cooled to 25° C. to 30° C. and quenched by slow addition of water. The layers are then separated and the organic layer is washed with water. Resulting organic layer is treated as a crude 1-bromo-2,4,5-trifluorobenzene. The obtained crude 1-bromo-2,4,5-trifluorobenzene is purified by fractional distillation to obtain pure 1-bromo-2,4,5-trifluorobenzene having purity of greater than 98.0%, preferably greater than 99.0% by GC.

The process for the preparation of 1-bromo-2,4,5-trifluorobenzene in one embodiment of the present invention is described herein after.

2,4,5-trifluoroaniline in the first step is converted into 1,2,4-trifluorobenzene which is then converted into 1-bromo-2,4,5-trifluorobenzene by any of the processes known in the literature.

In the first step, 2,4,5-trifluoroaniline is converted into 1,2,4-trifluorobenzene by reacting with deaminating agents such as sodium nitrite in the presence of a suitable per acid and mineral acid such as hydrogen chloride or sulphuric acid.

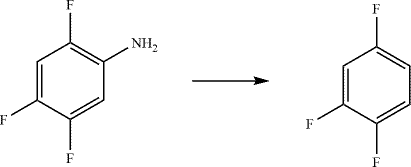 [SMILES: Nc1cc(F)c(F)cc1F ; Fc1ccc(F)c(F)c1]

Sodium nitrite used for the deamination is of concentration ranging from 10% to 40% by weight aqueous sodium nitrite, preferably concentration of aqueous sodium nitrite is 40% by weight.

Mineral acid such as hydrogen chloride or sulphuric acid used for the deamination is used in the concentration of 30% by weight aqueous hydrogen chloride or 30% by weight aqueous sulphuric acid.

Non-limiting examples of per acid suitable for converting 2,4,5-trifluoroaniline into 1,2,4-trifluorobenzene include hydrogen peroxide, peracetic acid, trifluoroperacetic acid, m-chloroperbenzoic acid, phthaloyl peroxide and 2,4-dinitroperbenzoic acid. Preferably, the per acid is 5% by weight aqueous hydrogen peroxide.

The conversion of 2,4,5-trifluoroaniline into 1,2,4-trifluorobenzene is carried out at a temperature ranging from 0° C. to100° C., preferably at a temperature ranging from 0° C. to 50° C., more preferably at a temperature ranging from 0° C. to 10° C.

The conversion of 2,4,5-trifluoroaniline into 1,2,4-trifluorobenzene is carried out for 0.5 hour to 10 hours, preferably for 1 hour to 6 hours.

After completion of the reaction, 10% by weight aqueous sodium hydroxide is added to the reaction mixture followed by addition of dichloromethane. Resulting mixture is filtered and the filtrate is allowed to settle to separate the layer. Resulting organic layer contains 1,2,4-trifluorobenzene. The intermediate 1,2,4-trifluorobenzene may or may not be isolated.

1,2,4-trifluorobenzene thus obtained is converted into 1-bromo-2,4,5-trifluorobenzene by any of the general processes reported for bromination of aromatic compounds or by the method as disclosed in Chinese patent CN101168495.

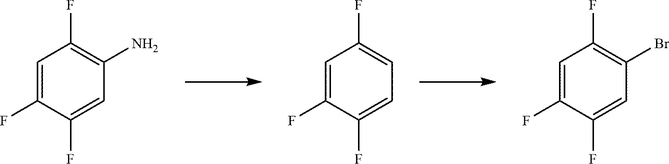 [SMILES: Nc1cc(F)c(F)cc1F ; Fc1ccc(F)c(F)c1 ; Fc1cc(F)c(Br)cc1F]

In another aspect of the present invention, provided is a process for preparation of an intermediate to prepare 1-bromo-2,4,5-trifluorobenzene, the process comprising converting 2,4,5-trifluoroaniline or sulfate salt thereof into an intermediate, wherein the intermediate is formed by reacting 2,4,5-trifluoroaniline or a sulfate salt thereof with nitrosulphuric acid to form 2,4,5-trifluorophenyl diazonium salt or by reacting 2,4,5-trifluoroaniline with sodium nitrite in the presence of mineral acid and per acid to form 1,2,4-trifluorobenzene.

In yet another aspect of the present invention 1-bromo-monohalobenzenes, 1-bromo-dihalobenzenes, and 1-bromo-trihalobenzenes are prepared from monohaloanilines, dihaloanilines and trihaloanilines respectively according to the processes of the present invention.

Various features and embodiments of the present invention are illustrated in the following representative examples, which are intended to be illustrative and non-limiting.

### EXAMPLES

### Example 1: Preparation of 1-bromo-2,4,5-trifluorobenzene

A mixture of 2,4,5-trifluoroaniline (5.0 g) and sulphuric acid (5.0 g) was brought in contact with nitrosulphuric acid and heated at 35° C. to 40° C. to obtain a reaction mixture comprising 2,4,5-trifluorophenyl diazonium salt. The reaction mixture was added to the solution of aqueous hydrogen bromide and copper (I) bromide at 100° C. to 105° C. and stirred for 2 hours. After completion of the reaction, the reaction was quenched by adding water. The reaction mass was extracted with methylene chloride to obtain 1-bromo-2,4,5-trifluorobenzene.

### Example 2: Preparation of 1,2,4-trifluorobenzene

2,4,5-trifluoroaniline (5 g) was added to 30% aqueous solution of hydrogen chloride (12.4 g) at 0° C. to 5° C. and stirred. After 30 minutes 5% hydrogen peroxide (10 g) was added and stirred further for 30 minutes. 40% aqueous solution of sodium nitrite was added slowly and stirred for 1 hour at 25° C. to 30° C. to obtain 1,2,4-trifluorobenzene.

### Example-3: Preparation of 1-bromo-2,4,5-trifluorobenzene

2,4,5-trifluoroaniline (42 g) was slowly added to concentrated sulphuric acid (98%, 42 g) at room temperature. Nitrosulphuric acid was added to the resulting mixture at room temperature over a period of 90 minutes and further stirred for 90 minutes at the same temperature to obtain a diazo solution.

After completion of the reaction, the resulting diazo solution was added to a stirred mixture of water, copper (I) bromide and aqueous hydrogen bromide at 90° C. to 105° C. over a period of 75 minutes. The reaction mixture was then stirred for 2.5 hours at the same temperature. After completion of the reaction, the reaction mixture was cooled and quenched with water. The layers were then separated, and the resulting organic layer contained a crude 1-bromo-2,4,5-trifluorobenzene having purity of 86.77% by GC.

### Example-4: Preparation of 1-bromo-2,4,5-trifluorobenzene

2,4,5-trifluoroaniline sulfate (300 g) was slowly added to nitrosulphuric acid at room temperature over a period of 60 minutes. The resulting mixture was then stirred for 3.0 hours at the same temperature to obtain a diazo solution.

After completion of the reaction, the resulting diazo solution was added to a stirred mixture of water, copper (I) bromide and aqueous hydrogen bromide at 45° C. to 75° C. over a period of 4 hours. The reaction mixture was then stirred for 3.0 hours. After completion of the reaction, the reaction mixture was cooled and quenched by slow addition of water. The layers were then separated, and the organic layer was washed with water. Resulting organic layer contained a crude 1-bromo-2,4,5-trifluorobenzene (214 g) having a purity of 95.28% by GC; Crude Yield: 82.8%.

Crude 1-bromo-2,4,5-trifluorobenzene was purified to obtain 188.9 g pure title compound having purity of more than 99.0% by GC; Yield: 73.1%

### Example-5: Preparation of 1-bromo-2,4,5-trifluorobenzene

2,4,5-trifluoroaniline sulfate (50 g) was slowly added to nitrosulphuric acid at room temperature over a period of 60 minutes. The resulting mixture was then stirred for 3.0 hours at the same temperature to obtain a diazo solution.

After completion of the reaction, the resulting diazo solution was added to a stirred mixture of acetonitrile, copper (I) bromide and aqueous hydrogen bromide at 50° C. to 75° C. over a period of 3.15 hours. The reaction mixture was then stirred for 3.0 hours at 50° C. to 75° C. After completion of the reaction, the reaction mixture was cooled and quenched with water. The layers were then separated. Resulting organic layer contained a crude 1-bromo-2,4,5-trifluorobenzene (35 g) having purity of 90.52% by GC; Yield: 81.3%.

### Example-6: Preparation of 1-bromo-2,4,5-trifluorobenzene

2,4,5-trifluoroaniline sulfate (50 g) was slowly added to nitrosulphuric acid at room temperature over a period of 60 minutes. The resulting mixture was then stirred for 3.0 hours at the same temperature to obtain a diazo solution.

After completion of the reaction, the resulting diazo solution was added to a stirred mixture of dimethyl sulfoxide, copper (I) bromide and aqueous hydrogen bromide at 50° C. to 75° C. over a period of 3.15 hours. The reaction mixture was then stirred for 3.0 hours at 50° C. to 75° C. After completion of the reaction, the reaction mixture was cooled and quenched by water. The layers were then separated. The resulting organic layer contained a crude 1-bromo-2,4,5-trifluorobenzene (35 g) having purity of 70.73% by GC; Yield: 81.3%

### Example-7: Preparation of 1-bromo-2,4,5-trifluorobenzene

2,4,5-trifluoroaniline sulfate (350 g) was slowly added to nitrosulphuric acid at room temperature over a period of 90 minutes. The resulting mixture was then stirred for 3.0 hours at the same temperature to obtain a diazo solution.

After completion of the reaction, the resulting diazo solution was added to a stirred mixture of copper (I) bromide and hydrogen bromide in acetic acid at 35° C. to 55° C. over a period of 6.0 hours. The reaction mixture was then stirred for 2.15 hours at 65° C. to 70° C. After completion of the reaction, the reaction mixture was cooled and quenched with water. The layers were then separated. The resulting organic layer contained a crude 1-bromo-2,4,5-trifluorobenzene having purity of 89.57% by GC.

### Example-8: Preparation of 1-bromo-2,4,5-trifluorobenzene

2,4,5-trifluoroaniline sulfate (100 g) was slowly added to nitrosulphuric acid at room temperature over a period of 60 minutes. The resulting mixture was then stirred for 3.0 hours at the same temperature to obtain a diazo solution.

After completion of the reaction, the resulting diazo solution was added to a stirred mixture of water, cupric oxide and aqueous hydrogen bromide at 50° C. to 75° C. over a period of 80 minutes. The reaction mixture was then stirred for 3.0 hours at 50° C. to 75° C. After completion of the reaction, the reaction mixture was cooled and quenched with water. The layers were then separated, and the organic layer was washed with water. The resulting organic layer contained a crude 1-bromo-2,4,5-trifluorobenzene (68 g) having purity of 95.35% by GC; Yield: 78.9%

### Example-9: Preparation of 1-bromo-2,4,5-trifluorobenzene

2,4,5-trifluoroaniline sulfate (100 g) was slowly added to nitrosulphuric acid at room temperature over a period of 60 minutes. The resulting mixture was then stirred for 3.0 hours at the same temperature to obtain a diazo solution.

After completion of the reaction, the resulting diazo solution was added to a stirred mixture of water, copper (II) bromide and aqueous hydrogen bromide at 50° C. to 75° C. over a period of 80 minutes. The reaction mixture was then stirred for 3.0 hours at 50° C. to 75° C. After completion of the reaction, the reaction mixture was cooled and quenched with water. The layers were then separated, and the organic layer was washed with water. The resulting organic layer contained a crude 1-bromo-2,4,5-trifluorobenzene (64 g) having a purity of 92.6% by GC; Yield: 74.3%.

### Example-10: Preparation of 1,2,4-trifluorobenzene

2,4,5-trifluoroaniline (4.48 g) was slowly added to 30% by weight aqueous solution of hydrogen chloride (12 g) at 0° C. to 5° C. The resulting mixture was then stirred for 30 minutes at the same temperature and 5% by weight aqueous hydrogen peroxide (9.0 g) was added slowly at 0° C. to 5° C. over a period of 90 minutes. The resulting mixture was then stirred for 30 minutes and 40% by weight aqueous sodium nitrite (6.2 g) was added to the mixture over a period of 60 minutes at same temperature. The resulting reaction mixture was then stirred for 60 minutes at 0° C. to 5° C. The reaction mixture was then heated to 25° C. to 30° C. and stirred further for 60 minutes. After completion of the reaction, 10% by weight aqueous sodium hydroxide (30 g) was added to the reaction mixture followed by addition of dichloromethane (200 g). The resulting mixture was filtered, and the filtrate was allowed to settle to separate the layer. The resulting organic layer contained 1,2,4-trifluorobenzene.

### Example-11: Preparation of 1,2,4-trifluorobenzene

2,4,5-trifluoroaniline (4.48 g) was slowly added to 30% by weight aqueous solution of sulphuric acid (32 g) at 0° C. to 5° C. The resulting mixture was then stirred for 30 minutes at the same temperature and 5% by weight aqueous hydrogen peroxide (9.0 g) was added slowly at 0° C. to 5° C. over a period of 90 minutes. The resulting mixture was then stirred for 30 minutes and 40% by weight aqueous sodium nitrite (6.2 g) was added to the mixture over a period of 60 minutes at the same temperature. The resulting reaction mixture was then stirred for 60 minutes at 0° C. to 5° C. The reaction mixture was then heated to 25° C. to 30° C. and stirred further for 60 minutes. After completion of the reaction, 10% by weight aqueous sodium hydroxide (65 g) was added to the reaction mixture followed by addition of dichloromethane (200 g). The resulting mixture was filtered, and the filtrate was allowed to settle to separate the layer. The resulting organic layer contained 1,2,4-trifluorobenzene.

### Comparative Example-1: Preparation of 1-bromo-2,4,5-trifluorobenzene

2,4,5-trifluoroaniline (250 g) was slowly added to 48% by weight aqueous hydrogen bromide (752 g) at 25° C. to 30° C. over a period of 30 minutes. The resulting mixture was then stirred for 30 minutes and water (700 g) was added at the same temperature. 40% by weight aqueous sodium nitrite (315.9 g) was then added to the resulting mixture at 0° C. to 5° C.

After completion of the reaction, the resulting diazo solution was added to a stirred solution of copper (I) bromide (7.0 g) and 48% by weight aqueous hydrogen bromide (125.37 g) at 0° C. to 5° C. over a period of 90 minutes. The reaction mixture was then stirred for 2.0 hours at 70° C. to 75° C. The reaction mixture was cooled to 25° C. to 30° C. to obtain polymerized reaction mass.

### Comparative Example-2: Preparation of 1-bromo-2,4,5-trifluorobenzene

2,4,5-trifluoroaniline (50 g) was slowly added to 25% by weight aqueous hydrogen chloride (111.65 g) at 25° C. to 30° C. over a period of 45 minutes. Water (94 g) was added to the resulting mixture at the same temperature. 40% by weight aqueous sodium nitrite (23.69 g) was then added to the resulting mixture at 0° C. to 5° C. over a period of 2.5 hours.

After completion of the reaction, the resulting diazo solution was slowly added to a stirred solution of copper (I) bromide (2.52 g) and 48% by weight aqueous hydrogen bromide (77.0 g) at 25° C. to 30° C. The reaction mixture was then stirred for 3.0 hours at 70° C. to 75° C. The reaction mixture was cooled to 25° C. to 30° C. to obtain a polymerized reaction mass.

### Comparative Example-3: Preparation of 1-bromo-2,4,5-trifluorobenzene

2,4,5-trifluoroaniline sulfate (79 g) was slowly added to 4.46% by weight aqueous sulphuric acid (282.0 g) at 25° C. to 30° C. over a period of 30 minutes. 40% by weight aqueous sodium nitrite (59.0 g) was then slowly added to the resulting mixture at -5° C. and stirred for 2.0 hours at the same temperature.

After completion of the reaction, the resulting diazo solution was slowly added to a stirred solution of copper (I) bromide (2.18 g) and 48% by weight aqueous hydrogen bromide (68.0 g) at 50° C. to 55° C. over a period of 35 minutes. The reaction mixture was then stirred for 2.0 hours at 70° C. to 75° C. The reaction mixture was cooled to 25° C. to 30° C. to obtain a polymerized reaction mass.

From the results of comparative examples 1 to 3, it was been observed that when 2,4,5-trifluoroaniline or 2,4,5-trifluoroaniline sulfate is diazotized using sodium nitrite and mineral acids such as hydrogen chloride or sulphuric acid followed by bromination using copper (I) bromide and hydrogen bromide results into polymerization of reaction mixture. Whereas, according to the process of the present invention diazotization of 2,4,5-trifluoroaniline or 2,4,5-trifluoroaniline sulfate using nitrosulphuric acid (instead of sodium nitrite) in sulphuric acid followed by bromination using suitable metal bromide and brominating agent provides the crude product yield in the range of 70% to 85% having purity in the range of 70% to 96% without polymerization of the reaction mass.

Thus, diazotization of 2,4,5-trifluoroaniline or 2,4,5-trifluoroaniline sulfate using nitrosulphuric acid followed by bromination using suitable metal bromide and brominating agent involves an inventive step and has huge industrial applicability.

The preparation of 1-bromo-2,4,5-trifluorobenzene from 2,4,5-trifluoroaniline or sulfate salt thereof using suitable reagents disclosed in the detailed description is novel as no such process involving specific reagent as shown in description is reported in the prior art.

The preparation of 1,2,4-trifluorobenzene from 2,4,5-trifluoroaniline is also novel and involves inventive-step.

Further, it shall be evident from the description that the preparation of 1-bromo-2,4,5-trifluorobenzene from 2,4,5-trifluoroaniline or sulfate salt thereof and the preparation of 1,2,4-trifluorobenzene from 2,4,5-trifluoroaniline involves an inventive step for the reason that the process is amenable to large scale, contributes to the technical advancement either through yield and/or purity or by obviating polymerization of reaction mass; and makes the commercial production safe and economical.

The embodiments herein and the various features and advantageous details thereof are explained with reference to the non-limiting embodiments in the description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments. Accordingly, the examples should not be construed as limiting the scope of the embodiments herein.

The description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

While considerable emphasis has been placed herein on the particular features of this invention, it will be appreciated that various modifications can be made, and that many changes can be made in the preferred embodiments without departing from the principles of the invention. These and other modifications in the nature of the invention or the preferred embodiments will be apparent to those skilled in the art from the invention herein, whereby it is to be distinctly understood that the foregoing descriptive matter is to be interpreted merely as illustrative of the invention and not as a limitation.

1. A process for the preparation of 1-bromo-2,4,5-trifluorobenzene comprising the steps of:
a) converting 2,4,5-trifluoroaniline or a sulfate salt thereof into an intermediate, wherein the intermediate is formed by reacting 2,4,5-trifluoroaniline or a sulfate salt thereof with nitrosulphuric acid or the intermediate is formed by reacting 2,4,5-trifluoroaniline with sodium nitrite in the presence of a mineral acid and a peracid; and
b) brominating the intermediate to obtain 1-bromo-2,4,5-trifluorobenzene 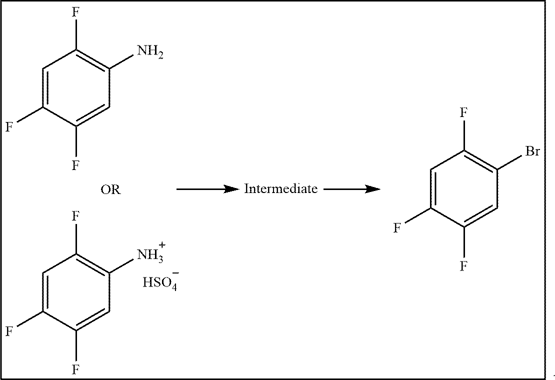
.

2. The process of claim 1, comprising reacting 2,4,5-trifluoroaniline or a sulfate salt thereof with nitrosulphuric acid to obtain 2,4,5-trifluorophenyl diazonium salt as the intermediate.

3. The process of claim 2, wherein the molar ratio of 2,4,5-trifluoroaniline or sulfate salt thereof and nitrosulphuric acid ranges from 1:1 to 1:1.5; and the concentration of nitrosulphuric acid in sulphuric acid ranges from 30% to 40% by weight.

4. The process of claim 1, comprising reacting 2,4,5-trifluoroaniline with sodium nitrite in the presence of mineral acid and peracid to obtain 1,2,4-trifluorobenzene as the intermediate.

5. A process for the preparation of 1-bromo-2,4,5-trifluorobenzene comprising the steps of:
a) diazotizing 2,4,5-trifluoroaniline or a sulfate salt thereof using nitrosulphuric acid to obtain 2,4,5-trifluorophenyl diazonium salt; and
b) brominating the 2,4,5-trifluorophenyl diazonium salt using a brominating agent in the presence of a metal bromide or in-situ generated metal bromide to obtain crude 1-bromo-2,4,5-trifluorobenzene, 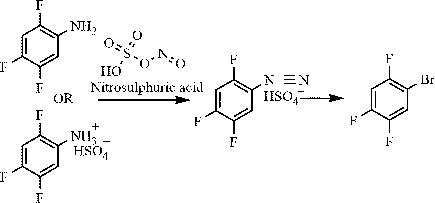
.

6. The process of claim 5, comprising purifying the crude 1-bromo-2,4,5-trifluorobenzene to obtain 1-bromo-2,4,5-trifluorobenzene having purity greater than 99%.

7. The process of claim 5, comprising using one or more solvents in step (b), wherein the one or more solvents is selected from the group consisting of acetonitrile, acetic acid, water, dimethyl sulfoxide, and mixtures thereof.

8. The process of claim 5, wherein the molar ratio of 2,4,5-trifluoroaniline or sulfate salt thereof and nitrosulphuric acid ranges from 1:1 to 1:1.5; and the concentration of nitrosulphuric acid in sulphuric acid ranges from 30% to 40% by weight.

9. The process of claim 5, wherein the metal bromide is selected from copper (I) bromide and copper (II) bromide; and the amount of metal bromide with respect to 2,4,5-trifluoroaniline or sulfate salt ranges from 2.5% to 3.0% by weight.

10. The process of claim 5, wherein the in-situ generated metal bromide is formed by reacting copper or cupric oxide with the brominating agent.

11. The process of claim 5 , wherein the brominating agent is hydrogen bromide or elemental bromine.

12. The process of claim 1 , wherein reacting 2,4,5-trifluoroaniline or a sulfate salt thereof with nitrosulphuric acid is carried out at a temperature ranging from 15° C. to 40° C. for 5 seconds to 5 hours; and the bromination reaction is carried out at a temperature ranging from 40° C. to 110° C. for 1 hour to 7 hours.

13. The process of claim 5, wherein the purity of crude 1-bromo-2,4,5-trifluorobenzene is in the range of 70% to 96%.

14. The process of claim 5, wherein the purity of crude 1-bromo-2,4,5-trifluorobenzene is in the range of 85% to 96%.

15. A process for the preparation of 1-bromo-2,4,5-trifluorobenzene comprising the steps of:
a) deaminating 2,4,5-trifluoroaniline in the presence of sodium nitrite, mineral acid and a peracid to obtain 1,2,4-trifluorobenzene; and
b) brominating 1,2,4-trifluorobenzene to obtain 1-bromo-2,4,5-trifluorobenzene, 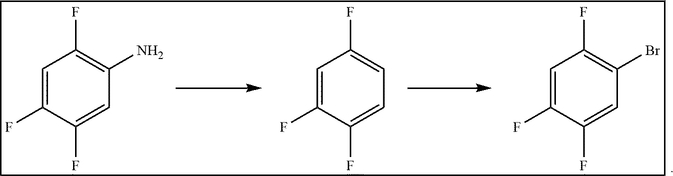
.

16. The process of claim 15, wherein the mineral acid is hydrogen chloride or sulphuric acid; and the peracid is selected from hydrogen peroxide, peracetic acid, and trifluoroperacetic acid.

17. The process of claim 15, wherein the deamination is carried out at a temperature ranging from 0° C. to 10° C. for 1 hour to 6 hours.

18. A process for the preparation of an intermediate to prepare 1-bromo-2,4,5-trifluorobenzene comprising:
converting 2,4,5-trifluoroaniline or sulfate salt thereof into an intermediate, wherein the intermediate is formed by reacting 2,4,5-trifluoroaniline or a sulfate salt thereof with nitrosulphuric acid to form 2,4,5-trifluorophenyl diazonium salt or by reacting 2,4,5-trifluoroaniline with sodium nitrite in the presence of a mineral acid and a peracid to form 1,2,4-trifluorobenzene.

19. The process of claims 10, wherein the brominating agent is hydrogen bromide or elemental bromine.

20. The process of claim 5, wherein reacting 2,4,5-trifluoroaniline or a sulfate salt thereof with nitrosulphuric acid is carried out at a temperature ranging from 15° C. to 40° C. for 5 seconds to 5 hours; and the bromination reaction is carried out at a temperature ranging from 40° C. to 110° C. for 1 hour to 7 hours.
